(19) United States
(12) Patent Application Publication
(10) Pub. No.: US 2021/0308093 A1
(43) Pub. Date: Oct. 7, 2021

(54) ACTIVATOR FOR SIMULTANEOUSLY ACTIVATING OXIDATIVE PHOSPHORYLATION AND INHIBITING GLYCOLYSIS AND USE THEREOF

(71) Applicant: LANZHOU UNIVERSITY, Lanzhou, Gansu (CN)

(72) Inventors: Jianye DAI, Lanzhou, Gansu (CN); Chu WANG, Lanzhou, Gansu (CN); Ning QIAO, Lanzhou, Gansu (CN)

(21) Appl. No.: 17/261,430

(22) PCT Filed: Jun. 28, 2020
(86) PCT No.: PCT/CN2020/000145

§ 371 Date: Jan. 19, 2021

(30) Foreign Application Priority Data
Jul. 4, 2019 (CN) 201910459038.8

(51) Int. Cl. A61K 31/352

(52) U.S. Cl. CPC A61K 31/352

(57) ABSTRACT

The present disclosure belongs to the technical field of molecular biology, and provides an activator for simultaneously activating oxidative phosphorylation and inhibiting glycolysis and use thereof in preparation of anti-cancer drugs, where the activator is baicalein. The present disclosure has the following beneficial effects: the present disclosure provides an activator for simultaneously activating oxidative phosphorylation and inhibiting glycolysis, that is, baicalein. This activator can activate the oxidative phosphorylation while inhibiting the glycolysis, thereby killing cancer cells. This mechanism can be a new anti-cancer mechanism, and key proteins involved in related pathways can be new targets for drug development.

This application claims priority of Chinese patent application No. 201910459038.8 filed to the China National Intellectual Property Administration (CNIPA) on Jul. 4, 2019 and entitled “ACTIVATOR FOR SIMULTANEOUSLY ACTIVATING OXIDATIVE PHOSPHORYLATION AND INHIBITING GLYCOLYSIS AND USE THEREOF”, which is herein incorporated by reference in its entirety.

### TECHNICAL FIELD

The present disclosure relates to the technical field of molecular biology and drug development, in particular to an activator for simultaneously activating oxidative phosphorylation and inhibiting glycolysis and use thereof.

### BACKGROUND

Oxidative phosphorylation is the metabolic pathway in which cells use enzymes to oxidize nutrients, thereby releasing the chemical energy stored within in order to produce adenosine triphosphate (ATP). This pathway is a coupling reaction which synthesizes ATP from adenosine diphosphate (ADP) and inorganic phosphoric acid using energy which is released during oxidization of substances in the body and supplied through a respiratory chain. This pathway is one of the most important metabolic in cells. 95% of ATP in organisms is generated in this way. However, mechanism of oxidative phosphorylation has been unclear all along. Moreover, the only small molecule compounds reported to regulate the pathway are inhibitors currently. Such substances which can block electron transfer at a certain site of the respiratory chain are called respiratory chain inhibitors. Among them, rotenone and amytal (or amobartital) inhibit electron transfer at nicotinamide adenine dinucleotide (NADH) dehydrogenase and block oxidation of NADH, but oxidation of flavin adenine dinucleotide (FADH2) can still proceed. Antimycin A inhibits electron transfer at cytochrome be 1 complex. Cyanide, carbon monoxide (CO), and azide (N3−) inhibit cytochrome oxidase. Substances that inhibit electron transfer and phosphorylation of ADP are called oxidative phosphorylation inhibitors, such as oligomycin. Moreover, 2,4-dinitrophenol (DNP) and valinomycin can decouple oxidation and phosphorylation, allowing electron transfer to proceed as usual without generating ATP. At the same time, cancer cells have a “Warburg effect”, that is, cancer cells tend to use glycolysis in place of oxidative phosphorylation which normal cells tend to use, so that cancer cells grow at a much faster rate than normal cells.

Natural products always play an important role in development of anti-cancer drugs. Both paclitaxel and camptothecin are anti-cancer stars (Zhou et al., 2016b). Rapamycin as a natural macrolide molecule even leads to discovery of a new mechanism (mTOR pathway), opening up a new era of drug discovery (Gopalakrishnan et al., 2018; Murray and Tee, 2018; Scott et al., 2009). It is noted that there are some natural products which exhibit unique biological activities in selectively killing cancer cells, for example, baicalein, an active ingredient in the traditional Chinese medicine Radix Scutellariae. According to existing literature, the baicalein has a broad spectrum anti-cancer effect, and shows a targeting anti-cancer activity in which glioma, breast cancer and other tumor cells can be specifically killed while corresponding normal cells are less likely to be toxicated (Parajuli et al., 2009; Zheng et al., 2014). Previous studies have also found that the baicalein has an anti-cancer activity on hepatocellular-carcinoma cells.

### SUMMARY

In view of the above defects in the prior art, a first objective of the present disclosure is to provide an activator for simultaneously activating oxidative phosphorylation and inhibiting glycolysis and use thereof in preparation of anti-cancer drugs.

In order to achieve the above objective, the present disclosure provides a technical solution: an activator for simultaneously activating oxidative phosphorylation and inhibiting glycolysis, where the activator is baicalein.

Further, for the above activator for simultaneously activating oxidative phosphorylation and inhibiting glycolysis, the baicalein may have a structure shown in formula I:

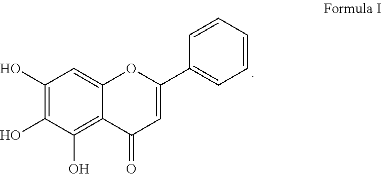 [SMILES: O=c1cc(-c2ccccc2)oc2cc(O)c(O)c(O)c12]

Further, for the above activator for simultaneously activating oxidative phosphorylation and inhibiting glycolysis, the baicalein may have a concentration of 100 μM.

A second objective of the present disclosure is to provide use of the above activator in preparation of anti-cancer drugs.

Based on a quantitative proteomics method, the present disclosure analyzes changes in a cancer cell protein network involved in an anti-cancer mechanism of baicalein, and finally discovers that the baicalein can activate oxidative phosphorylation and simultaneously inhibit glycolysis. Through knockdown experiments of key targets of oxidative phosphorylation, the application confirms activation of the oxidative phosphorylation which is expected to be a potential new anti-cancer mechanism where the key targets therein are promising to be new anti-cancer targets.

Working Principle and Working Process:

Cell and Animal Model Experiments of Baicalein for Anti-Cancer Activity

The present disclosure carries out experiments to test proliferation inhibition effects of baicalein on hepatocellular-carcinoma cell lines HuH7 and HepG2 respectively, with IC50 values calculated. Results show that, baicalein significantly inhibits the hepatocellular-carcinoma cell lines HuH7 and HepG2. Moreover, the present disclosure establishes a diethylnitrosamine-induced mouse liver cancer model which is then intervened with baicalein. Results show that baicalein has a significant therapeutic effect on mice with hepatocellular carcinoma, shows no liver toxicity on normal mice given the same dose of baicalein and causes no weight loss of mice. Therefore, baicalein can be considered safe to the liver and the whole system of mice while showing obvious anti-cancer activity.

Discovery of Effect of Baicalein on Oxidative Phosphorylation in an Anti-Cancer Process:

In order to clarify the anti-cancer mechanism of baicalein, a “panorama” is needed to find specific changes of protein network in hepatocellular-carcinoma cells intervened by baicalein. For this reason, the present disclosure uses stable isotope labeling by amino acids in cell culture (SILAC) technology for quantitative proteomics analysis. In simple terms, the SILAC technology uses medium containing heavy or light amino acids (arginine and lysine) for cell culture, and labels proteome with the heavy or light amino acids through cell metabolism. With “light labeled” and “heavy labeled” cells used for a control group experiment and a baicalein intervention group experiment, the quantitative proteomics method based on the SILAC technology can be used to reveal changes in the overall protein network caused by baicalein intervention in the hepatocellular-carcinoma cell line (HuH7) (see FIG. 2). The present disclosure finds that the most important baicalein affects is the oxidative phosphorylation.

Effect of baicalein on oxidative phosphorylation:

Based on the above results, effect of baicalein on oxidative phosphorylation needs to be clarified initially. Therefore, effects of different concentrations of baicalein on oxidative phosphorylation are tested at a cellular level. It is found that baicalein activates the oxidative phosphorylation in a concentration dependent manner (see FIG. 3). Further analysis finds that, baicalein (100 μM) can improve basal respiration value, maximum respiration value and ATP production in oxidative phosphorylation. It is noted that data show that, the present disclosure discovers the first natural activator of the oxidative phosphorylation. In order to further confirm effect of baicalein on the oxidative phosphorylation, the present disclosure further extracts mitochondria, a main site where the oxidative phosphorylation locates, and tests effect of baicalein concentration gradient and incubation time gradient on oxidative phosphorylation in the mitochondria. In consistent with the cell experiments, baicalein exhibits activation of the oxidative phosphorylation in a concentration dependent and time dependent manner. At this point, the present disclosure discovers a natural activator of the oxidative phosphorylation, the baicalein.

Effect of activation of oxidative phosphorylation by baicalein on anti-cancer mechanism:

With quantitative proteomics analysis, the present disclosure finds that the natural product baicalein can activate the oxidative phosphorylation. Then, it is necessary to clarify relationship between activation of the oxidative phosphorylation by baicalein and anti-cancer activity thereof. Cancer cells have the “Warburg effect”, that is, cancer cells tend to use glycolysis in replace of the oxidative phosphorylation which normal cells tend to use, so that cancer cells grow at a much faster rate than normal cells. It is found that baicalein inhibits the glycolysis while increasing level of the oxidative phosphorylation. The present disclosure verifies this phenomenon in HepG2 and HuH7 cells. It is found that, if a key enzyme of oxidative phosphorylation is knocked down by siRNA technology, the anti-cancer activity of baicalein is greatly weakened. (See FIG. 4)

The present disclosure has the following beneficial effects: the present disclosure provides an activator for simultaneously activating oxidative phosphorylation and inhibiting glycolysis, that is, baicalein. This activator can activate the oxidative phosphorylation while inhibiting the glycolysis, thereby killing cancer cells. This mechanism can be a new anti-cancer mechanism, and key proteins involved in related pathways can be new targets for drug development.

### BRIEF DESCRIPTION OF DRAWINGS

FIG. 1 shows anti-cancer effects of baicalein in cells and animal hepatocellular-carcinoma models, where

FIG. 1A shows a structure of baicalein;

FIG. 1B shows evaluation of dose dependent cytotoxicity of baicalein on hepatocellular-carcinoma cell lines HuH7 and HepG2 in an MTT (tetrazolium salt) colorimetric assay (n=6);

FIG. 1C shows hepatocellular-carcinoma pathological sections and body weights of normal and hepatocellular-carcinoma mice with or without baicalein; where “Saline” represents normal saline, “Baicalein” represents baicalein, and “IC50” represents median lethal dose.

FIG. 2 shows study results of anti-cancer mechanism of baicalein based on quantitative proteomics,

where

FIG. 2A is a schematic diagram of study of anti-cancer mechanism of baicalein based on quantitative proteomics;

FIG. 2B is a Venn diagram showing number of proteins identified in three experiments (shown in brackets);

FIG. 2C shows protein analysis, identifying proteins by the SILAC technology whose levels change more than one-fold in cancer cells in the baicalein intervention group compared with the group without baicalein intervention.

FIG. 3 shows effect of baicalein on oxidative phosphorylation, where

FIG. 3A shows effect of concentration gradient of baicalein on level of oxidative phosphorylation in HuH7 cells;

FIG. 3B shows that baicalein can significantly increase oxidative phosphorylation level of HuH7 cells in terms of, for example, basal respiration, maximum respiration, and ATP production;

FIG. 3C shows effects of baicalein concentration gradient and incubation time gradient on activation of oxidative phosphorylation in mitochondria; for all data, *p<0.05; **p<0.01; ***p<0.001, with baicalein administration group relative to dimethyl sulfoxide (DMSO) control group, n=6.

FIG. 4 shows effect of activation of oxidative phosphorylation by baicalein on anti-cancer mechanism thereof, where

FIG. 4A shows measurements that baicalein activates oxidative phosphorylation level while inhibiting glycolysis in HepG2 cells;

FIG. 4B shows measurements that baicalein activates oxidative phosphorylation level while inhibiting glycolysis in HuH7 cells;

FIG. 4C shows that knockdown of each of the eight key proteins in HuH7 cells with RNAi technology eliminates anti-cancer effect of baicalein in HuH7 cells to varying degrees; for all data, *p<0.05; **p<0.01; ***p<0.001, referring to a ratio of cell viability of baicalein administration group to that of DMSO control group, n=6.

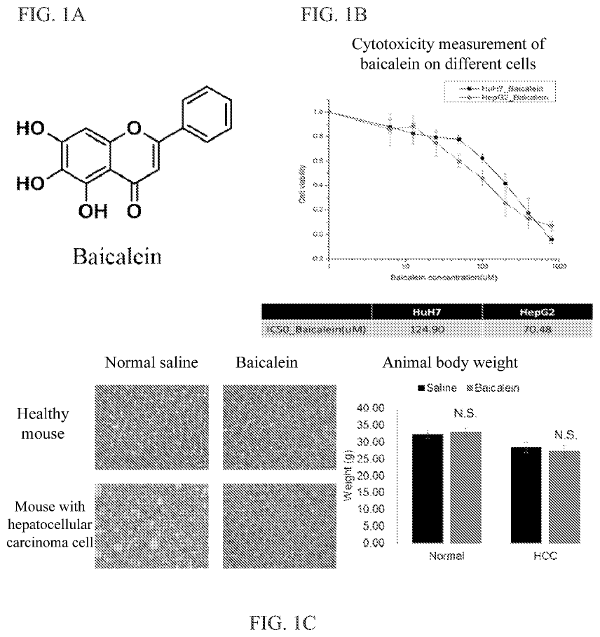

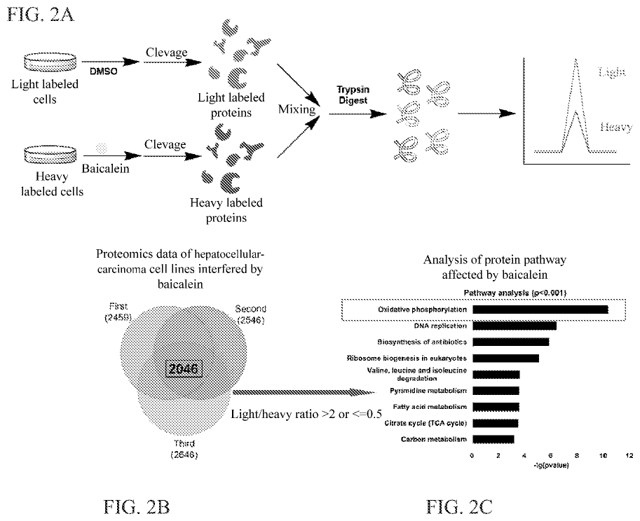

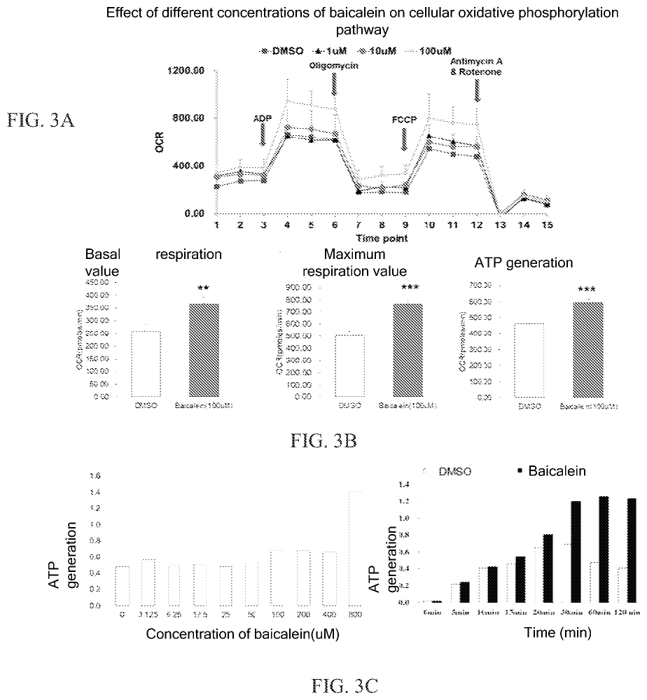

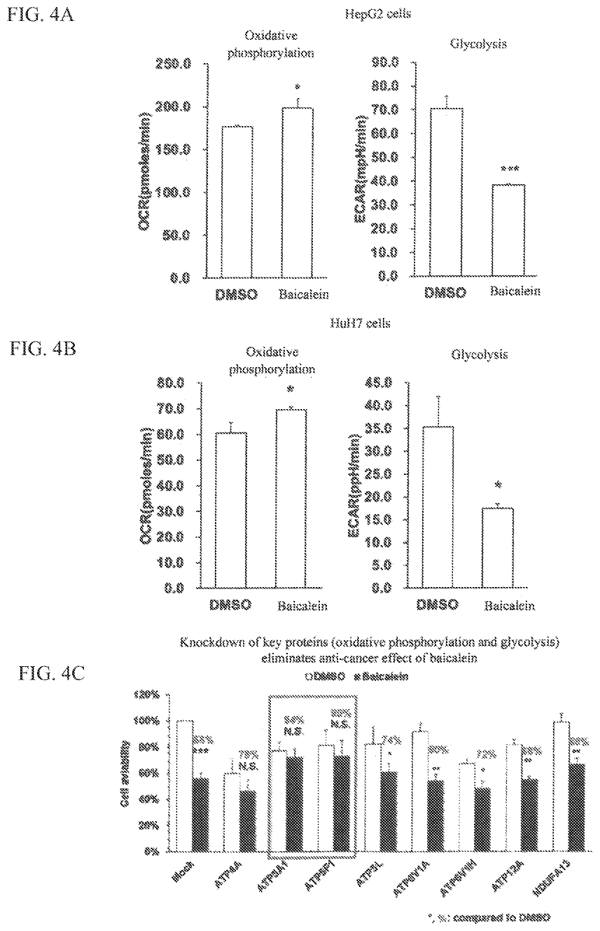

### DETAILED DESCRIPTION

### Example 1

An activator which can simultaneously activate oxidative phosphorylation and inhibit glycolysis was baicalein.

The baicalein had a structure shown in formula I:

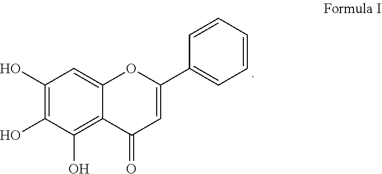 [SMILES: O=c1cc(-c2ccccc2)oc2cc(O)c(O)c(O)c12]

The baicalein had a concentration of 100 μM.

A second objective of the present disclosure was to provide use of the above activator in preparation of anti-cancer drugs.

Specific Verification Operations:

Cell Culture:

Hela, HepG2, and HuH7 cells (from China Center for Type Culture Collection (Wuhan, China)) were cultured at 37° C. with 5% CO₂ in Dulbecco's modified Eagle medium (DMEM, Thermo Fisher Scientific) supplemented with 10% fetal bovine serum (Thermo Fisher Scientific) and 1% penicillin-streptomycin (Thermo Fisher Scientific).

RNA Interference

The siRNA constructs listed below were designed and synthesized by GenePharma (Shanghai, China). At a modeling stage before baicalein treatment, interference was performed based on the RNAiMAX (Thermo Fisher Scientific) protocol.

MTT Assay:

10⁴ cells were added to a 96-well plate. 100 μL of pre-warmed medium was added to each well. After attachment, the cells were starved in a serum-free medium for 24 h, and then transferred to a normal medium containing 100 μM baicalein and cultured for another 24 h. After treatment, the cells were incubated with 100 μL of conventional medium containing 50 μg MTT (Sigma-Aldrich) for 4 h. Finally, purple precipitates were dissolved in 200 μL of DMSO (Sigma-Aldrich), and absorbance at 490 nm was measured by a microplate reader (Bio-Rad).

Measurement of Oxidative Phosphorylation:

Measurement of oxidative phosphorylation with Seahorse XF (Agilent): HuH7 cells, HepG2 cells and mitochondria were prepared. 1,000 cells or 4 μg mitochondria were added to the well plate provided by the Seahorse detection kit (Agilent, 103275-100). Related measurements were carried out following instructions.

Measurement of oxidative phosphorylation (ATP production) with QQQ-MS (AB Sciex): mitochondria were extracted from HuH7 cells. A reaction system was prepared according to Table 1 below. 4 μg of mitochondria was added to a 100 μL reaction system. Then different concentrations of baicalein were added. Incubation was carried out at 37° C. for corresponding time. Then 600 μL of cold methanol was added to stop the reaction and extraction of small molecules was carried out. At the same time, [¹³C]-ATP was added as an internal standard. After centrifugation at 20,000 g for 10 min, 400 μL of supernatant was drawn and added into 600 μL of secondary purified water. QQQ-MS analysis was carried out to calculate ATP production.

TABLE 1

| 70 mM | sucrose | 220 mM | mannitol |
|---|---|---|---|
| 0.2% (w/v) | BSA | 5 mM | MgCl₂ |
| 2 mM | HEPES | 1 mM | EGTA |

| | | |
|---|---|---|
| 2 mM | ADP | PBS solution |

SILAC Experiment:

The SILAC experiment was carried out with a protocol adapted from the ones previously reported (Martin B R et al., (2011) Nature methods 9(1):84-89; Weerapana E et al., (2007) Nature protocols 2(6):1414-1425). HuH7 cells were passaged in SILAC DMEM (Thermo Fisher Scientific) containing 10% SILAC FBS (Thermo Fisher Scientific), 1% penicillin-streptomycin (Thermo Fisher Scientific) and 100 μg/mL of [¹³C₆, ¹⁵N₄] L-arginine-HCl and [¹³C₆, ¹⁵N₂] L-lysine-HCl (Cambridge Isotope Laboratory) or L-arginine-HCl and L-lysine-HCl (Sigma-Aldrich).

Frozen cell pellets were resuspended in PBS containing 0.1% Triton X-100 (Sigma-Aldrich), sonicated and separated by ultracentrifugation at 100,000 g for 45 min into soluble and insoluble fractions. Concentration of soluble proteins was measured on a microplate reader (Bio-Rad) with BCA protein assay (Pierce™ BCA protein assay kit, Thermo Fisher Scientific). Enriched proteins were denatured in 6 M urea/PBS, reduced with 10 mM dithiothreitol (DTT, J&K Scientific) at 65° C. for 15 min, and blocked at 35° C. in the dark with 20 mM iodoacetamide (Sigma-Aldrich) for 30 min and stirred. Reactants were diluted with PBS to 2M urea/PBS. The supernatant was removed. Then, a pre-mixed solution of 100 mM calcium chloride aqueous solution and trypsin (20 μg, reconstituted in 40 μL of trypsin (Promega) buffer) was added, and stirred overnight at 37° C. Acidification was carried out with 5% formic acid the next day.

LC-MS/MS Analysis:

LC-MS/MS analysis was carried out on a Q-Exactive Orbitrap mass spectrometer (Thermo Fisher Scientific) coupled to Ultimate 3000LC system with a published protocol (5). In short, a flow rate through a column was set to be 0.3 μL/min, and applied remote spray voltage was set to be 2.8 kV. A full scan (350-1,800 MW) was used, followed by data-dependent MS2 scans for 20 most abundant ions by starting dynamic exclusion to collect MS2 data.

MS Data Analysis:

Peptide search was carried out using ProLuCID with variable modification of methionine (15.9949 Da), static modification of cysteine (57.0215 Da) and full trypsin specificity. Data were further filtered by DTASelect 2.0.47, and the false discovery rate was 1%. As mentioned above (Benjamin D I et al., (2012) Cell metabolism 16(5):565-577.), an internal software CIMAGE was used to quantify the SILAC ratio with minor modifications. Peptides whose chromatographic peaks were detected only in light samples but not in heavy samples were assigned a threshold ratio of 15 to reflect specificity enrichment. Only proteins with an average SILAC ratio (light/heavy) greater than 2.0 or less than 0.5 in all three replicates were selected for further GO analysis.

Animal Experiment:

All animal operations were carried out based on a protocol approved by the Animal Research Committee of Peking University, China, in accordance with the “Guidelines for Care and Use of Laboratory Animals” (NIH Publication No. 86-23, revised in 1985). All mice (C57BL/6j, Charles River, Beijing, China) were kept in a temperature-controlled barrier facility at Laboratory Animal Center in Peking University (laboratory animal facility approved by AAALAC) with a 12 h light/dark cycle, and had free access to food and water. Only male animals were used Animals were randomly grouped based on weight level. Five mice were selected for each group to meet requirements for statistical significance. A randomised, comparative, and single-blinded test was used. For wild littermates, modeling with diethylnitrosamine was started at 6 weeks of age and maintained for 24 weeks. After 12 weeks of modeling, 400 mg/kg baicalein (40 mg/mL saline) was administered by gavage daily for another 12 weeks, and then liver disease-related symptoms of these mice were analyzed.

Histological Analysis:

A liver sample cut from a same leaf of each animal was fixed in 4% paraformaldehyde overnight at room temperature, dehydrated with an ethanol gradient, infiltrated with xylene and embedded in paraffin. A paraffin embedded tissue was used to prepare serial sections 5 μm thick for hematoxylin and eosin (H&E) staining.

Statistics:

SPSS (IBM) was used to carry out Kolmogorov-Smirnov test and Levene test for normality and uniformity of variance of test data. When evaluating statistical significance of three or more means by comparison, one-way or two-way ANOVA was carried out with treatment or phenotype as an independent factor. When there was a statistical significance between two groups of measurements, two-sided Student's t-test was performed. P<0.05 was considered statistically significant. Unless otherwise stated, all data were expressed as mean±standard deviation.

Finally, it should be noted that the above descriptions are only preferred embodiments of the present disclosure and are not intended to limit the present disclosure. Although the present disclosure is described in detail with reference to the foregoing embodiments, a person skilled in the art can still make modifications to the technical solutions described in the foregoing embodiments, or make equivalent replacement to some technical features. Any modifications, equivalent substitutions, improvements and the like made within the spirit and scope of the present disclosure should be included within the protection scope of the present disclosure.

1. An activator for simultaneously activating oxidative phosphorylation and inhibiting glycolysis, wherein the activator is baicalein.

2. The activator according to claim 1, wherein the baicalein has a structure shown in formula I:

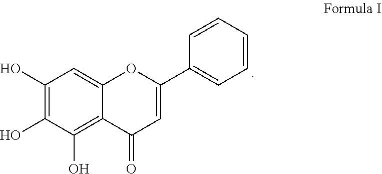 [SMILES: O=c1cc(-c2ccccc2)oc2cc(O)c(O)c(O)c12]

3. The activator according to claim 1, wherein the baicalein has a concentration of 100 μM.

4. An anti-cancer drug, comprising the activator according to claim 1 as an active compound.

5. An anti-cancer drug, comprising the activator according to claim 2 as an active compound.

6. An anti-cancer drug, comprising the activator according to claim 3 as an active compound.

7. A method for treating cancer, comprising administering an anti-cancer drug comprising the activator according to claim 1 to an individual in need thereof.

8. A method for treating cancer, comprising administering an anti-cancer drug comprising the activator according to claim 2 to an individual in need thereof.

9. A method for treating cancer, comprising administering an anti-cancer drug comprising the activator according to claim 3 to an individual in need thereof.
